(19) United States
(12) Patent Application Publication
(10) Pub. No.: US 2017/0079518 A1
(43) Pub. Date: Mar. 23, 2017

(54) METAL LARYNGOSCOPE BLADE WITH NON-METAL SAFETY TIP

(71) Applicant: TRUPHATEK INTERNATIONAL LTD., Netanya (IL)

(72) Inventors: Aviram ELBAZ, Ein Sarid (IL); Moshe KOHEN, Bat Yam (IL)

(21) Appl. No.: 15/125,072

(22) PCT Filed: Feb. 17, 2015
(86) PCT No.: PCT/IL2015/050177

§ 371 Date: Sep. 9, 2016

Related U.S. Application Data
(60) Provisional application No. 61/951,303, filed on Mar. 11, 2014.

(51) Int. Cl. A61B 1/267; A61B 1/00

(52) U.S. Cl. CPC A61B 1/267; A61B 1/0011

(57) ABSTRACT

A metal laryngoscope blade is provided for use in a laryngoscope including a laryngoscope handle to form an operative generally L-shaped configuration. The blade comprises a metal laryngoscope spatula having a longitudinal centerline, a proximate end and a distal end correspondingly adjacent to and remote from the laryngoscope handle in the operative generally L-shaped configuration, a top surface and a bottom surface correspondingly facing away from and towards the laryngoscope handle in the operative generally L-shaped configuration. The distal end has a leading safety tip contact zone including a front edge and opposite leading side edges meeting the front edge at leading corners. The blade further comprises a non-metal safety tip mounted on said distal end to embed said safety tip contact zone therein.

### FIELD OF THE INVENTION

The present disclosure is directed toward metal laryngoscope blades.

### BACKGROUND OF THE INVENTION

Metal laryngoscope blades include a metal laryngoscope spatula with a brazed metal tip typically made of stainless steel and brazed using silver or similar inert metals. Brazing is a relatively cumbersome and time consuming manufacturing process. Moreover, brazing can lead to the formation of cavities at a metal tip which trap water leading to corrosion. Moreover, brazed metal tips can injure soft tissues particularly during a difficult intubation or an emergency intubation.

U.S. Pat. No. 3,826,248 to Gobels entitled Laryngoscope discloses a spatula including an insert of elastic material, for example, rubber, plastic, or the like, for elastic reception of a patient's teeth when the spatula rests against the teeth upon use of the laryngoscope.

### SUMMARY OF THE INVENTION

The present disclosure is directed towards metal laryngoscope blades having a non-metal safety tip as opposed to a conventional brazed metal tip. The non-metal safety tip can be formed from a wide range of non-metal materials including inter alia silicon, plastic, rubber, polymer, for example, polyurethane (PU), and the like. The non-metal safety tips can be mounted on a laryngoscope spatula by means of conventional manufacturing processes including inter alia overmolding, gluing, ultrasonic welding, interference fit mounting, and the like. The non-metal safety tips reduce injuries to soft tissues and facilitate less traumatic intubations compared to brazed metal tips. The non-metal safety tips can be color coded for facilitating visual distinction between different sizes of laryngoscope blades.

Laryngoscope blades with a non-metal safety tip of the present invention can be so-called Macintosh blades with a curved spatula, English style Macintosh blades, Miller blades with a straight spatula, and the like. Laryngoscope blades including a non-metal safety tip of the present invention can be constituted as a discrete metal laryngoscope blade intended for detachable mounting on a discrete laryngoscope handle. Commonly owned U.S. Pat. No. 7,736,304 entitled Metal Laryngoscope Blade discloses such laryngoscope blades. Alternatively, metal laryngoscope blades with a non- metal safety tip of the present invention can be constituted as a single complete laryngoscope including a laryngoscope handle and an integral non-detachable laryngoscope blade. Commonly owned U.S. Pat. No. 7,909,759 entitled Handheld Penknife-Like Laryngoscope discloses such laryngoscopes.

### BRIEF DESCRIPTION OF THE DRAWINGS

In order to understand the presently disclosed subject matter and to see how it may be carried out in practice, a preferred embodiment will now be described, by way of a non-limiting example only, with reference to the accompanying drawings, in which:

FIG. 1 is a pictorial view of a conventional dissembled dual component laryngoscope including a laryngoscope handle and a metal laryngoscope blade having a metal laryngoscope spatula with a brazed metal tip;

FIG. 2 is a side view of the FIG. 1 metal laryngoscope blade;

FIG. 3 is an enlarged side elevation view of the distal end of the FIG. 2 laryngoscope spatula denoted A;

FIG. 4 is a pictorial view of a dissembled dual component laryngoscope including a laryngoscope handle and a metal laryngoscope blade having a metal laryngoscope spatula with a non-metal safety tip in accordance with the present invention;

FIG. 5 is an exploded view of the distal end of the FIG. 4 metal laryngoscope blade;

FIG. 6 is a side elevation view of the FIG. 4 metal laryngoscope blade;

FIG. 7 is an enlarged side elevation view of the distal end denoted B in FIG. 6;

FIG. 8 is a top plan view of the distal end of the FIG. 4 metal laryngoscope blade; and

FIG. 9 is a longitudinal cross section view of the distal end of the FIG. 4 metal laryngoscope blade along line 9-9 in FIG. 8.

### DETAILED DESCRIPTION OF THE DRAWINGS

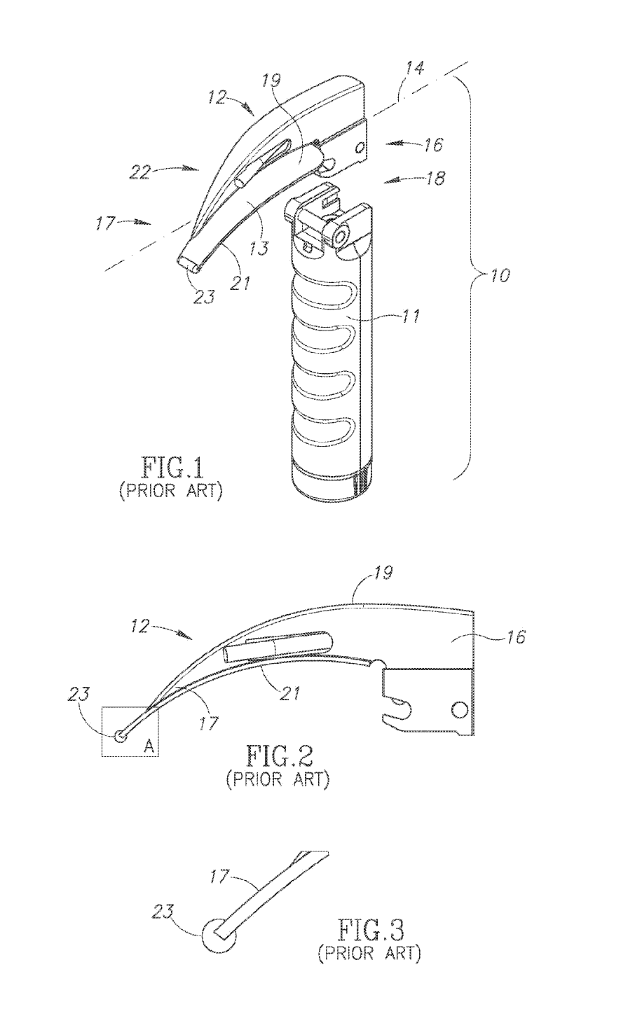

FIGS. 1 to 3 show a conventional dual component laryngoscope 10 including a laryngoscope handle 11 and a metal laryngoscope blade 12 in an operative generally L-shaped configuration for assisting intubation. The metal laryngoscope blade 12 includes a laryngoscope spatula 13 having a longitudinal centerline 14 and a proximate end 16 and a distal end 17 correspondingly adjacent to and remote from the laryngoscope handle 11 in the operative generally L-shaped configuration. The laryngoscope 10 includes a standard double snap fit arrangement 18 for detachable dual snap fit engagement of the metal laryngoscope blade 12 on the laryngoscope handle 11. The metal laryngoscope spatula 13 includes a top surface 19 and a bottom surface 21 correspondingly facing away from and towards the laryngoscope handle 11 in the operative generally L-shaped configuration.

The dual component laryngoscope 10 includes a conventional illumination arrangement 22 for providing illumination at the distal end 17 in the operative generally L-shaped configuration. Conventional illumination arrangements 22 include either a blade mounted illumination source or a handle mounted illumination source and a fiber optic light guide for transmitting illumination light from the handle mounted illumination source to the distal end 17. The distal end 17 terminates in a brazed metal tip 23. The metal laryngoscope blade 12 can be optionally formed with a teeth protector as described hereinabove with reference to U.S. Pat. No. 3,826,248 to Gobels.

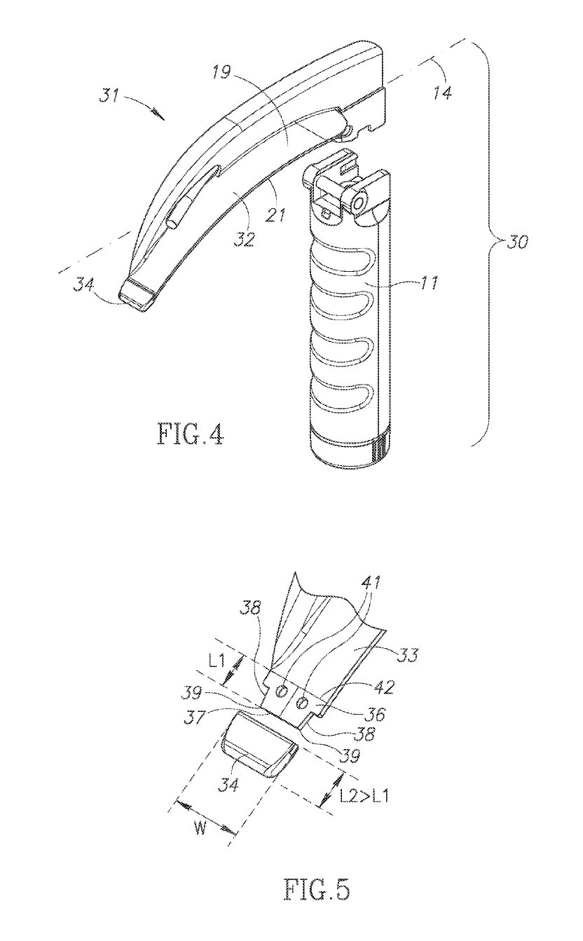

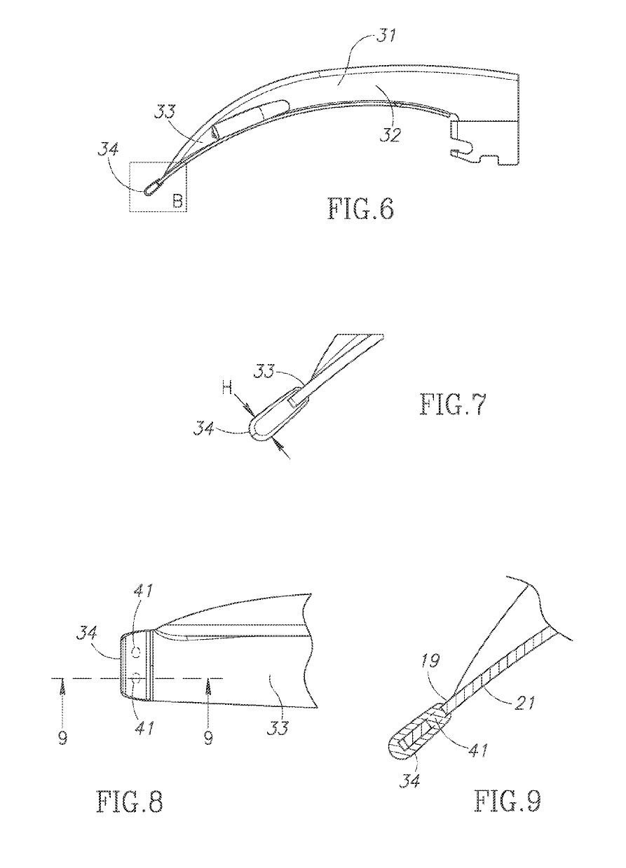

FIGS. 4 to 9 show a dual component laryngoscope 30 similar in construction and use as the dual component laryngoscope 10 and therefore similar parts are likewise numbered. The latter 30 differs from the former 10 insofar as the latter 30 includes a metal laryngoscope blade 31 having a metal laryngoscope spatula 32 with a distal end 33 and a non-metal safety tip 34 instead of the brazed metal tip 23. The non-metal safety tip 34 can be formed from a wide range of non-metal materials including inter alia silicon, PU, plastic, rubber, and the like. The non-metal safety tip 34 preferably has a Shore hardness value in the range of 30 to 120 for the different materials.

The distal end 33 has a leading safety tip contact zone 36 having a front edge 37 and opposite leading side edges 38 meeting the front edge 37 at leading corners 39. The safety tip contact zone 36 is formed with one or more throughgoing bores 41 extending between the top surface 19 and the bottom surface 21. The non-metal safety tip 34 extends through the one or more throughgoing bores 41 for mechanically securing same on the safety tip contact zone 36. The safety tip contact zone 36 has a length L1 of at least 4 mm from the front edge 37 along the longitudinal centerline 14 to an imaginary line 42 trailing behind the one or more throughgoing bores 41.

The non-metal safety tip 34 can be mounted on the metal laryngoscope spatula 32 by means of conventional manufacturing processes including inter alia overmolding, gluing, ultrasonic welding, interference fit mounting, and the like. The non-metal safety tip 34 entirely embeds the safety tip contact zone 36 therein to afford wraparound protection during an intubation to prevent soft tissues injuries in contradistinction to the use of brazed metal tips 23.

Non-metal safety tips 34 are preferably color coded for indicating the size of a metal laryngoscope blade 31. Non-metal safety tips 34 are typically proportional in size to the size of a metal laryngoscope blade 21 in terms of their width W, length L2 where L2>L1 and height H. Exemplary Small, Medium and Large sizes of non-metal safety tips 34 have the following exemplary dimensions:

| | Small (mm) | Medium (mm) | Large (mm) |
|---|---|---|---|
| Width | 10.0 | 12.0 | 15.0 |
| Length | 6.0 | 7.2 | 8.3 |
| Height | 3.0 | 3.2 | 3.2 |

While the invention has been described with respect to a limited number of embodiments, it will be appreciated that many variations, modifications, and other applications of the invention can be made within the scope of the appended claims.

1. A metal laryngoscope blade for use in a laryngoscope including a laryngoscope handle to form an operative generally L-shaped configuration, the blade comprising:
(a) a metal laryngoscope spatula having a longitudinal centerline, a proximate end and a distal end correspondingly adjacent to and remote from the laryngoscope handle in the operative generally L-shaped configuration, and
a top surface and a bottom surface correspondingly facing away from and towards the laryngoscope handle in the operative generally L-shaped configuration,
said distal end having a leading safety tip contact zone including a front edge and opposite leading side edges meeting said front edge at leading corners; and
(b) a non-metal safety tip mounted on said distal end to embed said safety tip contact zone therein.

2. The blade according to claim 1 wherein said safety tip contact zone includes at least one throughgoing bore extending between said top surface and said bottom surface and said non-metal safety tip extends through said at least one throughgoing bore.

3. The blade according to claim 2 wherein said non-metal safety tip is color coded for indicating the size of the metal laryngoscope blade.

4. A laryngoscope comprising a laryngoscope handle and a non-detachable metal laryngoscope blade with a non-metal safety tip according to claim 3.

5. The blade according to claim 1 wherein said non-metal safety tip is color coded for indicating the size of the metal laryngoscope blade.

6. A laryngoscope comprising a laryngoscope handle and a non-detachable metal laryngoscope blade with a non-metal safety tip according to claim 5.

7. A laryngoscope comprising a laryngoscope handle and a non-detachable metal laryngoscope blade with a non-metal safety tip according to claim 1.

8. A laryngoscope comprising a laryngoscope handle and a non-detachable metal laryngoscope blade with a non-metal safety tip according to claim 2.
